SEQUENCE LISTING (27 sequences)

SEQ ID NO: 1 - DNA, 26 bp, Artificial Sequence
  Description of Artificial Sequence Synthetic oligonucleotide
gtgagtcagactgcgacagttcgagt

SEQ ID NO: 2 - DNA, 26 bp, Artificial Sequence
  Description of Artificial Sequence Synthetic oligonucleotide
actcgaacagtcgcagtctgactcac

SEQ ID NO: 3 - DNA, 25 bp, Artificial Sequence
  Description of Artificial Sequence Synthetic oligonucleotide
ttaatttggatttggaaacgagagt

SEQ ID NO: 4 - DNA, 25 bp, Artificial Sequence
  Description of Artificial Sequence Synthetic oligonucleotide
actctcgtttccaaatccaaattaa

SEQ ID NO: 5 - DNA, 26 bp, Artificial Sequence
  Description of Artificial Sequence Synthetic oligonucleotide
accccacgcgcttggaagcgcaggat

SEQ ID NO: 6 - DNA, 26 bp, Artificial Sequence
  Description of Artificial Sequence Synthetic oligonucleotide
atcctgcgcttccaagcgcgtggggt

SEQ ID NO: 7 - DNA, 26 bp, Artificial Sequence
  Description of Artificial Sequence Synthetic oligonucleotide
gcctgaactggagactagctgtgaat

SEQ ID NO: 8 - DNA, 26 bp, Artificial Sequence
  Description of Artificial Sequence Synthetic oligonucleotide
attcacagctagtctccagttcaggc

SEQ ID NO: 9 - DNA, 26 bp, Artificial Sequence
  Description of Artificial Sequence Synthetic oligonucleotide
atgctgttttgcgttttccggggggt

SEQ ID NO: 10 - DNA, 26 bp, Artificial Sequence
  Description of Artificial Sequence Synthetic oligonucleotide
accccccggaaaacgcaaaacagcat

SEQ ID NO: 11 - DNA, 26 bp, Artificial Sequence
  Description of Artificial Sequence Synthetic oligonucleotide
ccgatcctagacaaatgtggaagagt

SEQ ID NO: 12 - DNA, 26 bp, Artificial Sequence
  Description of Artificial Sequence Synthetic oligonucleotide
actcttccacatttgtctaggatcgg

SEQ ID NO: 13 - DNA, 26 bp, Artificial Sequence
  Description of Artificial Sequence Synthetic oligonucleotide
tcacgcttactacaacccatcagagt

SEQ ID NO: 14 - DNA, 26 bp, Artificial Sequence
  Description of Artificial Sequence Synthetic oligonucleotide
actctgatgggttgtagtaagcgtga

SEQ ID NO: 15 - DNA, 26 bp, Artificial Sequence
  Description of Artificial Sequence Synthetic oligonucleotide
gcattagtaagtttgatctggagaat

SEQ ID NO: 16 - DNA, 26 bp, Artificial Sequence
  Description of Artificial Sequence Synthetic oligonucleotide
attctccagatcaaacttactaatgc

SEQ ID NO: 17 - DNA, 26 bp, Artificial Sequence
  Description of Artificial Sequence Synthetic oligonucleotide
acaggagtggaaaccctcgactggat

SEQ ID NO: 18 - DNA, 26 bp, Artificial Sequence
  Description of Artificial Sequence Synthetic oligonucleotide
atccagtcgagggtttccactcctgt

SEQ ID NO: 19 - DNA, 20 bp, Artificial Sequence
  Description of Artificial Sequence Synthetic oligonucleotide
tcgagtctgaagcgagagct

SEQ ID NO: 20 - DNA, 20 bp, Artificial Sequence
  Description of Artificial Sequence Synthetic oligonucleotide
gagagtttctggtcatgaaa

SEQ ID NO: 21 - DNA, 20 bp, Artificial Sequence
  Description of Artificial Sequence Synthetic oligonucleotide
caggatgggaaaagaaggtg

SEQ ID NO: 22 - DNA, 20 bp, Artificial Sequence
  Description of Artificial Sequence Synthetic oligonucleotide
gtgaatctccagcagaggga

SEQ ID NO: 23 - DNA, 20 bp, Artificial Sequence
  Description of Artificial Sequence Synthetic oligonucleotide
ggggggtctcctctaaccac

SEQ ID NO: 24 - DNA, 20 bp, Artificial Sequence
  Description of Artificial Sequence Synthetic oligonucleotide
aagagtgataggactctatg

SEQ ID NO: 25 - DNA, 20 bp, Artificial Sequence
  Description of Artificial Sequence Synthetic oligonucleotide
cagagtccctaattacaatc

SEQ ID NO: 26 - DNA, 20 bp, Artificial Sequence
  Description of Artificial Sequence Synthetic oligonucleotide
gagaatgaagctctgattac

SEQ ID NO: 27 - DNA, 20 bp, Artificial Sequence
  Description of Artificial Sequence Synthetic oligonucleotide
ctggatggagcaattgggaa

(19) United States
(12) Patent Application Publication
(10) Pub. No.: US 2019/0093091 A1
(43) Pub. Date: Mar. 28, 2019

(54) COMPOSITIONS FOR ERADICATING FLAVIVIRUS INFECTIONS IN SUBJECTS

(71) Applicant: Temple University - of the Commonwealth System of Higher Education, Philadelphia, PA (US)

(72) Inventors: Kamel Khalili, Bala Cynwyd, PA (US); Hassen Wollebo, Philadelphia, PA (US)

(21) Appl. No.: 16/091,874

(22) PCT Filed: Mar. 29, 2017
(86) PCT No.: PCT/US2017/024769

§ 371 Date: Oct. 5, 2018

Related U.S. Application Data
(60) Provisional application No. 62/319,106, filed on Apr. 6, 2016.

(51) Int. Cl. C12N 9/22; C12N 15/11; A61K 38/46

(52) U.S. Cl. CPC C12N 9/22; C12N 2310/20; A61K 38/465; C12N 15/11

(57) ABSTRACT

Compositions that specifically cleave target sequences in Flavivirus, for example Zika virus, include nucleic acids encoding a Clustered Regularly Interspaced Short Palindromic Repeat (CRISPR) associated endonuclease and a guide RNA sequence complementary to a target sequence in a Zika virus. These compositions are administered to a subject for treating an infection or at risk for contracting a Zika virus infection.

### FIELD OF THE INVENTION

The present invention relates to compositions that specifically cleave target sequences in Flavivirus, for example, Zika virus. Such compositions, which include nucleic acids encoding a Clustered Regularly Interspaced Short Palindromic Repeat (CRISPR) associated endonuclease and a guide RNA sequence complementary to a target sequence in a Zika virus, can be administered to a subject having or at risk for contracting a Zika virus infection.

### BACKGROUND

Once a rare virus found in the rhesus monkey in the Zika forest in Uganda, the Zika virus has become an urgent public health concern in many countries and has been associated with microcephaly in neonates and Guillain-Barré syndrome in adults (Dick et al. 1952. Trans R Soc Trap Med Hyg 46: 509-520; Broutet et al., 2016, N Engl J Med (In Press); Chan et al., 2016, J Infect (In Press); Lazear and Diamond, 2016, J Virol JVI.00252-16 (In Press); Vogel, 2016 Science 351: 1123-1124). The virus remained obscure with few human cases confined to Africa and Asia (Moore et al., 1975, Ann Trap Med Parasitol 69: 49-64) until the Asian strain caused Zika outbreaks in Micronesia in 2007 (Haddow et al., 2012, Bull World Health Organ 31: 57-69) & French Polynesia in 2013-2014 (Cao-Lormeau et al., 2014, Emerg Infect Dis 20: 1085-1086).

In French Polynesia (2013-2014), the outbreak spread to other Pacific Islands: New Caledonia, Cook Islands, Easter Island, Vanuatu, and Solomon Islands (Musso D. 2015, Emerg Infect Dis 21: 1887). Zika virus then spread to Brazil by an unknown means of transmission but phylogenetic studies showed that closest strain to the one that emerged in Brazil was from samples from French Polynesia and spread in the Pacific Islands (Campos et al., 2015, Emerg Infect Dis 21: 1885-1886; Musso, D. 2015, Emerg Infect Dis 21: 1887). The first report of autochthonous Zika transmission in the Americas was in March 2015 in Rio Grande do Norte, Northeast Brazil (Zanluca et al., 2015; Hennessey et al., 2016). The epidemic has spread in Brazil with now ˜1,300,000 suspected cases in late 2015 (Hennessey et al., 2016, MMWR Morb Mortal Wkly Rep 65: 55-58; Bogoch et al., 2016, Lancet 387: 335-336). Already Zika has begun to spread beyond Brazil and further spread of is anticipated with imported cases already been reported in the US, Europe and other countries where travelers are returning after visiting Latin America and the Caribbean (Hennessey et al, 2016, MMWR Morb Mortal Wkly Rep 65: 55-58; Hills et al., 2016, MMWR Morb Mortal Wkly Rep 65: 215-216).

The rapid advance of the virus and the reported high rates of microcephaly and Guillain-Barré syndrome associated with Zika infection in Polynesia and Brazil have raised concerns that it represents an evolving neuropathic and teratogenic public health threat. The Pan American Health Organization predicts that Zika virus will spread to eventually reach all areas where Aedes mosquitoes are endemic (Malone et al., 2016, PLoS Negl Trop Dis 10: e0004530). There are no licensed vaccines, therapeutic or preventive drugs available for Zika virus and hence the development and deployment of countermeasures are urgently needed.

Ominously, it now appears that the virus may be able to be transmitted by means other than the Aedes mosquito (Lazear and Diamond, 2016, J Virol JVI.00252-16 (In Press)). Firstly, since Zika is a bloodborne pathogen, it is possible that a Zika-infected blood donor could contaminate the blood supply and cases of Zika transmission through transfusion have been reported in Brazil (Lazear and Diamond, 2016). The efficiency of the transmission of Zika virus by transfusions is still unknown and additional studies are needed (Musso et al., 2014, Euro Surveill 19(14) pii: 20761; Marano et al., 2016, Blood Transfus 14: 95-100). Screening of donated blood by PCR-based tests as is done for West Nile Virus would prevent this possibility if these become available or, if not, application of strategies for inactivation of the virus (Kleinman, S. 2015, Curr Opin Hemnatol 22: 547-553; Aubry et al., 2016, Transfusion 56: 33-40). Secondly, Zika can be transmitted sexually (Foy et al., 2011, Emerg Infect Dis 17: 880-882; Musso et al., 2015, Emerg Infect Dis 21: 1887; Hills et al., 2016, MMWR Morb Mortal Wkly Rep 65: 215-216) and in these cases, virus was transmitted from infected men to their female partners. Accordingly, Zika viral RNA can be detected in semen (Musso et al., 2015, Emerg Infect Dis 21: 1887; Mansuy et al., 2016, Lancet Infect Dis (In Press)) and in one report, the RNA virus load was about 100,000 times that of matched blood or urine samples at a time of more than 2 weeks after the onset of symptoms. Lastly, perinatal transmission of Zika has been reported but it is not known if this occurred in utero, via breast milk or by a bloodborne route (Besnard et al., 2014, Euro Surveill 19(13) pii: 20751). This may be particularly important given the association of Zika with neonatal abnormalities such as microcephaly.

### SUMMARY

Embodiments of the invention are directed to compositions for eradicating a Flavivirus, in vitro or in vivo. Methods of treatment or prevention of an infection comprises the use of the compositions.

In some embodiments, a composition for eradicating a flavivirus in vitro or in vivo, comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome. In some embodiments, the target nucleic acid sequence comprises one or more nucleic acid sequences in coding and non-coding nucleic acid sequences of the Flavivirus genome.

In an embodiment of the invention, the target nucleic acid sequence comprises one or more sequences within a sequence encoding structural proteins, non-structural proteins or combinations thereof. The sequences encoding structural proteins comprise nucleic acid sequences encoding a capsid protein (C), precursor viral membrane protein (prM), viral membrane protein (M), envelop protein (E) or combinations thereof. The sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

In some embodiments, a gRNA has at least a50%, 60%, 65% or at least 75% sequence identity to a nucleic acid sequence that is complementary to target nucleic acid sequences encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof. In some embodiments, the gRNA sequences have at least a 50%, 60%, 65% or at least 75% sequence identity to sequences comprising: SEQ ID NO: 1-18, or combinations thereof. In other embodiments, the gRNA sequences comprise: SEQ ID NO: 1-18, or combinations thereof.

In some embodiments, the composition comprises a short proto-spacer adjacent motif (PAM)-presenting DNA oligonucleotide sequence (PAMmer) wherein the PAMmer comprises a PAM and additional Flavivirus nucleic acid sequences downstream of target Flavivirus nucleic acid sequences of the gRNA. In some embodiments, a PAMmer oligonucleotide sequence comprises a nucleic acid sequence having at least a 50%, 60%, 65% or at least 75% sequence identity to at least one nucleic acid sequence comprising: SEQ ID NOS: 19-27, or combinations thereof. In other embodiments, the PAMmer has at least one nucleic acid sequence comprising SEQ ID NOS: 19-27, or combinations thereof.

In other embodiments, an isolated nucleic acid sequence encodes a Clustered Regularly Interspaced Short Palindromic Repeat (CRISPR)-associated endonuclease and at least one guide oligonucleotide, the guide oligonucleotide being complementary to a target nucleic acid sequence in a Flavivirus genome. The target nucleic acid sequence comprises one or more nucleic acid sequences in coding and non-coding nucleic acid sequences of the Flavivirus genome. In some embodiments, the target nucleic acid sequence comprises one or more sequences within a sequence encoding structural proteins, non-structural proteins or combinations thereof. In embodiments, the sequences encoding structural proteins comprise nucleic acid sequences encoding a capsid protein (C), precursor viral membrane protein (prM), viral membrane protein (M), envelop protein (E) or combinations thereof. The sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

In some embodiments, the guide oligonucleotide sequence has at least a 50%, 60%, 65% or at least 75% sequence identity to a nucleic acid sequence that is complementary to target nucleic acid sequences encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

In certain embodiments, a gRNA sequence has at least a 50%, 60%, 65% or at least 75% sequence identity to a nucleic acid sequence that is complementary to, or having at least a 50%, 60%, 65% or at least 75% sequence identity to target nucleic acid sequence encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

In some embodiments, the guide oligonucleotide sequences have a 50%, 60%, 65% or at least 75% sequence identity to at least one sequence comprising: SEQ ID NO: 1-27, or any combinations thereof. In other embodiments, the guide oligonucleotide sequences comprise: SEQ ID NO: 1-27, or combinations thereof. In other embodiments, the isolated nucleic acid sequences further comprise a short proto-spacer adjacent motif (PAM)-presenting DNA oligonucleotide sequence (PAMmer) wherein the PAMmer comprises a PAM and additional Flavivirus nucleic acid sequences downstream of target Flavivirus nucleic acid sequences of the gRNA. In some embodiments, PAMmer oligonucleotide sequence has at least one nucleic acid sequence comprising SEQ ID NOS: 19-27, or combinations thereof.

In other embodiments, a vector comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

In another embodiment, a composition comprises a vector encoding an isolated nucleic acid sequence encoding a gene editing agent and/or at least one guide oligonucleotide, the guide oligonucleotide being complementary to a target nucleic acid sequence in a Flavivirus genome.

In some embodiments, the gene editing agent and the at least one guide oligonucleotide is encoded by the same vector. In other embodiments, a first vector encodes the gene editing agent and a second vector encodes at least one guide oligonucleotide, the guide oligonucleotide being complementary to a target nucleic acid sequence in a Flavivirus genome. In yet another embodiment, a vector encodes at least two or more guide oligonucleotides, the guide oligonucleotides being complementary to the same target nucleic acid sequences and/or different target nucleic acid sequences. In yet another embodiment, a vector encodes one or more gene editing agents. In yet another embodiment a vector encodes one or more gene editing agents, two or more gene editing agents, three or more gene editing agents, and/or one or more guide oligonucleotides, two or more guide oligonucleotides, three or more guide oligonucleotides, or any number and combination of gene editing agents and/or guide oligonucleotides.

In some embodiments, a gene editing agent comprises Clustered Regularly Interspaced Short Palindromic Repeat (CRISPR)-associated endonuclease, Argonaute family of endonucleases, zinc-finger nucleases (ZFNs), transcription activator-like effector nucleases (TALENs), meganucleases, other endo- or exo-nucleases, or combinations thereof. In some embodiments, the gene editing agent comprises Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments or combinations thereof.

In other embodiments, a method of eradicating a Flavivirus genome in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

In yet another embodiment, a method of inhibiting replication of a Flavivirus in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

Other aspects are described infra.

### BRIEF DESCRIPTION OF THE DRAWINGS

FIGS. 1A and 1B show the identification of targets for gene editing within the genome of Zika virus of 10,794 nucleotides in size. FIG. 1A: The positions of the untranslated terminal repeats (UTR) repeat at the 5′-end (nucleotides 1-106) and the 3′-end (nucleotides 10367-10794) are shown. Two target sequences 5′-M1 (nucleotides 14 to 34) and 5′-M2 (nucleotides 72 to 91), each containing 6 nucleotides, PAM sequence within the 5′-UTR, and three target sequences, 3′-M1 (nucleotides 10542 to 10568), 3′-M2 (nucleotides 10609 to 10635), and 3′-M3 (nucleotides 10664 to 10690) for the creation of gRNAs, are depicted. FIG. 1B: The positions of the DNA coding sequence corresponding to NS3 and NS5 genes within the Zika genome, each with two target sequences for the creation of gRNAs, NS3-M1 (nucleotides 5012 to 5037), NS3-M2 (nucleotides 5402 to 5428), NS5-M1 (nucleotides 9279 to 9304), NS5-M2 (nucleotides 9733 to 9758) are shown. The positions of the 6 nucleotide PAM sequence are highlighted at the 3′ ends of each target sequence.

### DETAILED DESCRIPTION

Embodiments of the invention are directed to compositions for eradicating a flavivirus, in vitro or in vivo. In particular, the compositions comprise isolated nucleic acid sequences encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “eradication” of the Flavivirus, e.g. Zika virus, as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context. An oligonucleotide is specifically hybridizable when there is a sufficient degree of complementarity to avoid non-specific binding of the oligonucleotide to non-target nucleic acid sequences under conditions in which specific binding is desired. Such conditions include, i.e., physiological conditions in the case of in vivo assays or therapeutic treatment, and conditions in which assays are performed in the case of in vitro assays.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, primates, rodents including mice, rats, and hamsters.

The term “polynucleotide” is a chain of nucleotides, also known as a “nucleic acid” or “nucleic acid sequence” and include, but are not limited to, all nucleic acid sequences which are obtained by any means available in the art, both naturally occurring and synthetic nucleic acids, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The nucleic acid sequences may be “chimeric,” that is, composed of different regions.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “percent sequence identity” or having “a sequence identity” refers to the degree of identity between any given query sequence and a subject sequence.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Compositions for Eradication of Flavivirus in Cells or Subjects

Zika virus is related to other human flaviviruses that cause significant pathology including yellow fever, dengue, tick-borne encephalitis, Saint Louis encephalitis, Japanese encephalitis and West Nile viruses and is most closely related to Spondweni virus (Faye et al., 2014, PLoS Negl Trop Dis 8(1): e2636). Like other flaviviruses, the genome is about 11 Kb in length and expresses seven nonstructural proteins and three structural proteins that are encoded as a single polyprotein in a unique long open reading frame containing all of the structural protein genes at the 5′ portion of the genome and the nonstructural (NS) protein genes at the 3′ portion. The genome organization of flaviviruses, concerning the protein expression order is:

5′-C-prM-E-NS1-NS2a-NS2b-NS3-NS4a-NS4b-NS5-3′

The capsid protein (C) is 13 kDa in size, highly basic and complexes with the viral RNA in the nucleocapsid while the outer membrane of the virion is a lipid bilayer containing the viral membrane protein (M) and envelope protein (E). The M protein is expressed as a larger glycosylated precursor protein (prM) while the E protein may or may not be glycosylated and this is a determinant of neuroinvasion, acting to increase both axonal and trans-epithelial transportation (Neal, 2014, J Infect 69: 203-215). The genomic RNA of flaviviruses lacks a poly-A tail at the 3′ end (Wengler and Wengler, 1981, Virology 13: 544-555) and has an m⁷gpppAmpN₂ at the 5′ end (Cleaves and Dubin, 1979, Virology 96: 159-165). Several regions within the genome of flaviviruses have a highly conserved structure including a 90-120 nucleotide stretch near the 3′ end, which is thought to form a stable hairpin loop (Brinton et al., 1986, Virology 153: 113-121). Mutational analysis of this region in Dengue virus revealed that it has an essential role in viral replication (Zeng et al., 1998, J Virol 72: 7510-7522).

Flavivirus particles bind to the surface of target cells by interactions between viral surface glycoproteins and cellular cell surface receptors. Virions undergo receptor-mediated endocytosis and are internalized into clathrin-coated pits (Gollins and Porterfield, 1985, J Gen Virol 66: 1969-1982). Uncoating of the virus envelope releases the viral RNA into the cytoplasm and also activates the host cell innate response followed by complex interplay between virus and host where virus co-opts the host cytoplasmic membranes for replication of its genome and the host attempts to control infection with several responses including interferon release, the unfolded protein/endoplasmic reticulum response, autophagy and apoptosis (Nain et al., 2016, Rev Med Virol 26: 129-141). Translation of viral proteins from the viral RNA occurs from the long open reading frame to produce a large polyprotein that is cleaved co- and posttranslationally into the individual viral proteins and leads to replication of the viral genome.

The viral RNA, structural and non-structural proteins and some host proteins are involved in the assembly of the viral replication complex in vesicle packages in the cytoplasm of infected cells (Lindenbach and Rice, 2003, Adv Virus Res 59: 23-61). Replication initiates with the synthesis of a negative-strand RNA, which then serves as a template for the synthesis of copies of the positive-strand genomic RNA in an asymmetric fashion such that there is 10- to 100-fold excess of positive strands over negative strands (Cleaves et al., 1981, Virology 111: 73-83). Replication requires the activities of several of the viral nonstructural (NS) proteins. NS3 consists of an N-terminal serine protease and a C-terminal helicase with NS3 protease activity requiring NS2B as a cofactor, and cleaving the viral polyprotein at several positions between the NS proteins. The NS3 helicase domain has helicase, RNA-stimulated nucleoside triphosphate hydrolase and 5′-RNA triphosphatase activities with the helicase activity required for unwinding the double-stranded RNA intermediate formed during genome synthesis and the 5′-RNA triphosphatase activity required for 5′-RNA cap formation. NS5 contains a C-terminal RNA-dependent RNA polymerase (RdRp) activity that is involved in viral genome replication and carries out both (−) and (+) strand RNA synthesis (Klema et al., 2015, Viruses 7: 4640-4656). Virus particles assemble by budding into the endoplasmic reticulum and nascent virus particles traverse the host secretory pathway, where virion maturation occurs followed by release from the cell (Lindenbach and Rice, 2003, Adv Virus Res 59: 23-61). Zika virus can be cultured in suckling mice and also grows well in Vero cells (Way et al., 1976, J Gen Virol 30: 123-130). In infections in vivo, flaviviruses can target a variety of cell types including dendritic cells, macrophages, endothelial cells and neuronal cells (Hidari and Suzuki, 2011, Trop Med Health 39(4 Suppl): 37-43; Dalrymple and Mackow, 2014, Curr Opin Virol 7:134-140; Neal, 2014, J Infect 69: 203-215).

No clinically approved therapy is currently available for the treatment of Zika or indeed any other flavivirus infection (Lim et al., 2013, Antiviral Res 100: 500-519). Over the past decade, significant effort has been made towards dengue drug discovery. Due to the similarity between Zika virus and dengue virus, it is possible that knowledge from dengue drug discovery could be applied to Zika virus. Several approaches are possible, e.g., high-throughput screening using virus replication assays or viral enzyme assays, structure-based in silico docking and rational design strategies and repurposing hepatitis C virus inhibitors for Zika. The development of antivirals should focus on distinctive features of Zika molecular biology that can be exploited. For example, Zika NS3 protein has a protease activity that is necessary for the viral life cycle and this may be a viable target for small molecule antiviral inhibitors. In this regard, the inhibitors of the NS3/4A protease of Hepatitis C, telaprevir and boceprevir, revolutionize the management of hepatitis C genotype 1 patients (Vermehren and Sarrazin, 2011, Eur J Med Res 16: 303-314). NS3 also has a 5′-RNA triphosphatase activity required for 5′-RNA cap formation and NS5 contains a C-terminal RNA-dependent RNA polymerase (RdRp) activity as described above and these are also potential targets for the development of small molecule antiviral inhibitors (Lim et al., 2015, Antiviral Res 100: 500-519; Luo et al., 2015, Antiviral Res 118: 148-158). Finally, the advent of methodologies such as the CRISPR/Cas9 system that are specifically able to target nucleotide sequences within viral genomes has provided an effective, specific, and versatile weapon against human DNA viruses (White et al., 2015, Discov Med 19: 255-262).

Gene Editing Agents:

Compositions for eradication of a Flavivirus include using a guided gene editing agent which is specifically targeted to viral nucleic acid sequences for destruction and eradication of that virus in a host cell in vitro or in vivo. Any suitable gene editing agent can be used, such as, nuclease systems including, for example, the Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference.

In embodiments, the compositions disclosed herein, include nucleic acids encoding a CRISPR-associated endonuclease, such as Cas9. In some embodiments, one or more guide RNAs that are complementary to a target sequence of a Flavivirus may also be encoded.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains.

In embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

Three types (I-m) of CRISPR systems have been identified. CRISPR clusters contain spacers, the sequences complementary to antecedent mobile elements. CRISPR clusters are transcribed and processed into mature CRISPR RNA (crRNA). In embodiments, the CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately. The term “guide RNA” (gRNA) will be used to denote either a crRNA:tracrRNA duplex or an sgRNA. It will be understood the term “gRNA complementary to” a target sequence indicates a gRNA whose spacer sequence is complementary to the target sequence.

The CRISPR-associated endonuclease Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium dificle, Finegoldia magna, Natranaerobius thermophilus, Pelotomaculun thermopropionicum, Acidithiobacillus caldus, Acidithiobacillusferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Pseudomonas aeruginosa, Escherichia coli, or other sequenced bacteria genomes and archaea, or other prokaryotic microorganisms may also be a source of the Cas9 sequence utilized in the embodiments disclosed herein.

In some embodiments, the CRISPR-associated endonuclease can be a sequence from another species, for example, other bacterial species, bacteria genomes and archaea, or other prokaryotic microorganisms. Alternatively, the wild type Cas9, CasX, CasY.1, CasY.2, CasY.3, CasY.4, CasY.5, CasY.6, ARMAN 1, ARMAN 4, sequences can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GI:669193757; KM099232.1 GI:669193761; or KM099233. GI:669193765. Alternatively, the Cas9, CasX, CasY.1, CasY.2, CasY.3, CasY.4, CasY.5, CasY.6, ARMAN 1, ARMAN 4, sequences can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765, or Cas9 amino acid sequence of PX330 or PX260 (Addgene, Cambridge, Mass.).

The wild type Streptococcus pyogenes Cas9, the CasX, CasY.1, CasY.2, CasY.3, CasY.4, CasY.5, CasY.6, ARMAN 1, ARMAN 4, sequences can be a mutated sequence. For example, the Cas9 nuclease can be mutated in the conserved HNH and RuvC domains, which are involved in strand specific cleavage. In another example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The sequences of Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof, can be modified to encode biologically active variants, and these variants can have or can include, for example, an amino acid sequence that differs from a wild type by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, polypeptides can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9, CasX, CasY.1, CasY.2, CasY.3, CasY.4, CasY.5, CasY.6, spCas, eSpCas, SpCas9, ARMAN 1, ARMAN 4 polypeptides. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

In addition to the wild type and variant Cas9 endonucleases described, embodiments of the invention also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI: 10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9(K848A/K1003A/R 1060A) (a.k.a. eSPCas9 1.1) are employed in the compositions. The invention is by no means limited to these variants, and encompasses all Cas9 variants (Slaymaker, I. M. et al. (2015)). The present invention also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D 1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple, and quadruple combinations of N497A, R661A, Q695A, Q926A or any other substitutions (Kleinstiver, B. P. et al., 2016, Nature. DOI: 10.1038/nature 16526).

In one embodiment, the endonuclease is derived from a type II CRISPR/Cas system. In other embodiments, the endonuclease is derived from a Cas9 protein and includes Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof.

Two nucleic acids or the polypeptides they encode may be described as having a certain degree of identity to one another. For example, a Cas9 protein and a biologically active variant thereof may be described as exhibiting a certain degree of identity. Alignments may be assembled by locating short Cas9 sequences in the Protein Information Research (PIR) site (pir.georgetown.edu), followed by analysis with the “short nearly identical sequences” Basic Local Alignment Search Tool (BLAST) algorithm on the NCBI website (ncbi.nlm.nih.gov/blast).

A percent sequence identity to Cas9 can be determined and the identified variants may be utilized as a CRISPR-associated endonuclease and/or assayed for their efficacy as a pharmaceutical composition. A naturally occurring Cas9 can be the query sequence and a fragment of a Cas9 protein can be the subject sequence. Similarly, a fragment of a Cas9 protein can be the query sequence and a biologically active variant thereof can be the subject sequence. To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chenna et al., Nucleic Acids Res. 31:3497-3500, 2003.

As used herein, the term “Cas” is meant to include all Cas molecules comprising variants, mutants, orthologues, high-fidelity variants, and the like.

In some embodiments, the endonuclease comprises Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments or combinations thereof. The endonucleases may be the same or may vary. For example, one endonuclease may be a Cas9, another endonuclease may be CasY.5 or ARMAN 4 and the like. Accordingly, the isolated nucleic acid sequence can encode any number and type of endonuclease.

Other CRISPR systems that can be used include CRISPR/Cpf1, which is a DNA-editing technology analogous to the CRISPR/Cas9 system, characterized in 2015 by Feng Zhang's group from the Broad Institute and MIT. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations.

Argonaute proteins can also be used. Argonaute proteins are proteins of the PIWI protein superfamily that contain a PIWI (P element-induced wimpy testis) domain, a MID (middle) domain, a PAZ (Piwi-Argonaute-Zwille) domain and an N-terminal domain. Argonaute proteins are capable of binding small RNAs, such as microRNAs, small interfering RNAs (siRNAs), and Piwi-interacting RNAs. Argonaute proteins can be guided to target sequences with these RNAs in order to cleave mRNA, inhibit translation, or induce mRNA degradation in the target sequence. There are several different human Argonaute proteins, including AGO1, AGO2, AGO3, and AGO4 that associate with small RNAs. AGO2 has slicer ability, i.e. acts as an endonuclease. Argonaute proteins can be used for gene editing. Endonucleases from the Argonaute protein family (from Natronobacterium gregoryi Argonaute) also use oligonucleotides as guides to degrade invasive genomes. The Natronobacterium gregoryi Argonaute (NgAgo) is a DNA-guided endonuclease suitable for genome editing in human cells. NgAgo binds 5′ phosphorylated single-stranded guide DNA (gDNA) of ˜24 nucleotides, efficiently creates site-specific DNA double-strand breaks when loaded with the gDNA. The NgAgo-gDNA system does not require a protospacer-adjacent motif (PAM), as does Cas9, and preliminary characterization suggests a low tolerance to guide-target mismatches and high efficiency in editing (G+C)-rich genomic targets. The Argonaute protein endonucleases used in the present invention can also be Rhodobacter sphaeroides Argonaute (RsArgo). RsArgo can provide stable interaction with target DNA strands and guide RNA, as it is able to maintain base-pairing in the 3′-region of the guide RNA between the N-terminal and PIWI domains. RsArgo is also able to specifically recognize the 5′ base-U of guide RNA, and the duplex-recognition loop of the PAZ domain with guide RNA can be important in DNA silencing activity. Other prokaryotic Argonaute proteins (pAgos) can also be used in DNA interference and cleavage. The Argonaute proteins can be derived from Arabidopsis thaliana, D. melanogaster, Aquifex aeolicus, Thennus thermophiles, Pyrococcusfuriosus, Thermus thermophilus JL-18, Thermus thermophilus strain HB27, Aquifex aeolicus strain VF5, Archaeoglobus fulgidus, Anoxybacillus flavithermus, Halogeometricum borinquense, Microsystis aeruginosa, Clostridium bartlettii, Halorubrum lacusprofundi, Thermosynechococcus elongatus, and Synechococcus elongatus. Argonaute proteins can also be used that are endo-nucleolytically inactive but post-translational modifications can be made to the conserved catalytic residues in order to activate them as endonucleases. Therefore, the present invention also provides for a pharmaceutical composition including at least one isolated nucleic acid sequence encoding at least one Argonaute protein, which targets at least one nucleotide sequence of a flavivirus genome, the isolated nucleic acid sequences being included in at least one expression vector. This composition can further include any of siRNA, miRNAs, shRNAs, or RNAi further described below.

Human WRN is a RecQ helicase encoded by the Werner syndrome gene. It is implicated in genome maintenance, including replication, recombination, excision repair and DNA damage response. These genetic processes and expression of WRN are concomitantly upregulated in many types of cancers. Therefore, it has been proposed that targeted destruction of this helicase could be useful for elimination of cancer cells. Reports have applied the external guide sequence (EGS) approach in directing an RNase P RNA to efficiently cleave the WRN mRNA in cultured human cell lines, thus abolishing translation and activity of this distinctive 3′-5′ DNA helicase-nuclease. RNase P RNA is another potential endonuclease for use with the present invention.

The Class 2 type VI-A CRISPR/Cas effector “C2c2” demonstrates an RNA-guided RNase function. C2c2 from the bacterium Leptotrichia shahii provides interference against RNA phage. In vitro biochemical analysis show that C2c2 is guided by a single crRNA and can be programmed to cleave ssRNA targets carrying complementary protospacers. In bacteria, C2c2 can be programmed to knock down specific mRNAs. Cleavage is mediated by catalytic residues in the two conserved HEPN domains, mutations in which generate catalytically inactive RNA-binding proteins. The RNA-focused action of C2c2 complements the CRISPR-Cas9 system, which targets DNA, the genomic blueprint for cellular identity and function. The ability to target only RNA, which helps carry out the genomic instructions, offers the ability to specifically manipulate RNA in a high-throughput manner and manipulate gene function more broadly.

Another Class 2 type V-B CRISPR/Cas effector “C2c1” can also be used in the present invention for editing DNA. C2c1 contains RuvC-like endonuclease domains related distantly to Cpf1. C2c1 can target and cleave both strands of target DNA site-specifically. According to Yang, et al. (Cell, 2016 Dec. 15; 167(7):1814-1828)), a crystal structure confirms Alicyclobacillus acidoterrestris C2c1 (AacC2c1) binds to sgRNA as a binary complex and targets DNAs as ternary complexes, thereby capturing catalytically competent conformations of AacC2c1 with both target and non-target DNA strands independently positioned within a single RuvC catalytic pocket. C2c1-mediated cleavage results in a staggered seven-nucleotide break of target DNA, crRNA adopts a pre-ordered five-nucleotide A-form seed sequence in the binary complex, with release of an inserted tryptophan, facilitating zippering up of 20-bp guide RNA:target DNA heteroduplex on ternary complex formation, and that the PAM-interacting cleft adopts a “locked” conformation on ternary complex formation.

C2c3 is a gene editor effector of type V-C that is distantly related to C2c1, and also contains RuvC-like nuclease domains. C2c3 is also similar to the CasY.1-CasY.6 group described below.

A CRISPR/TevCas9 system can also be used. In some cases, it has been shown that once CRISPR/Cas9 cuts DNA in one spot, DNA repair systems in the cells of an organism will repair the site of the cut. The TevCas9 enzyme was developed to cut DNA at two sites of the target so that it is harder for the cells' DNA repair systems to repair the cuts (Wolfs, et al., PNAS, doi: 10.1073). The TevCas9 nuclease is a fusion of an I-Tevi nuclease domain to Cas9.

The gene editor or gene editing agent can also be Archaea Cas9. The size of Archaea Cas9 is 950 amino acid ARMAN 1 and 967 amino acid ARMAN 4. The Archaea Cas9 can be derived from ARMAN-1 (Candidatus Micrarchaeum acidiphilun ARMAN-1) or ARMAN-4 (Candidatus Parvarchaeum acidiphilum ARMAN-4).

The gene editing agent can also be CasX. CasX has a TTC PAM at the 5′ end (similar to Cpf1 1). The TTC PAM can have limitations in viral genomes that are GC rich, but not so much in those that are GC poor. The size of CasX (986 bp), smaller than other type V proteins, provides the potential for four gRNA plus one siRNA in a delivery plasmid. CasX can be derived from Deltaproteobacteria or Planctomycetes.

Guide RNA Sequences:

The compositions and methods of the present invention may include a sequence encoding a guide RNA that is complementary to a target sequence in a flavivirus, e.g., Zika virus. Guide RNA sequences according to the present invention can be sense or anti-sense sequences. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3 and Neiseria meningitidis requires 5′-NNNNGATT. The specific sequence of the guide RNA may vary, but, regardless of the sequence, useful guide RNA sequences will be those that minimize off-target effects while achieving high efficiency and complete ablation of the Flavivirus, for example, the Zika virus. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

In another preferred embodiment, a guide oligonucleotide comprises combinations of phosphorothioate internucleotide linkages and at least one internucleotide linkage selected from the group consisting of: alkylphosphonate, phosphorodithioate, alkylphosphonothioate, phosphoramidate, carbamate, carbonate, phosphate triester, acetamidate, carboxymethyl ester, and/or combinations thereof.

In another preferred embodiment, a guide oligonucleotide optionally comprises at least one modified nucleobase comprising, peptide nucleic acids, locked nucleic acid (LNA) molecules, analogues, derivatives and/or combinations thereof.

The compositions and methods of the present invention may include a sequence encoding a guide RNA that is complementary to a target sequence in a Flavivirus. Examples of Flaviviruses include, for example: Dengue Fever Virus, West Nile Fever Virus, Yellow Fever Virus, St. Louis Encephalitis Virus, Japanese Encephalitis Virus, Murray Valley Encephalitis Virus, Tick-borne Encephalitis Virus, Kunjin Encephalitis Virus, Rocio Encephalitis Virus, Russian Spring Summer Encephalitis Virus, Negishi Virus, Kyasanur Forest Virus, Omsk Hemorrhagic Fever Virus, Powassan Virus, Louping III Virus, Rio Bravo Virus, Tyuleniy Virus, Ntaya Virus, Modoc Virus, Alkhurma Hemorrhagic Fever Virus, Zika virus. In one embodiment, the Flavivirus is Zika virus.

In certain embodiments, a composition for eradicating a flavivirus in vitro or in vivo, comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome. In certain embodiments, the isolated nucleic acid sequence further comprises a short proto-spacer adjacent motif (PAM)-presenting DNA oligonucleotide sequence (PAMmer). As used herein the “PAMmer” is an oligonucleotide comprising a PAM and additional Flavivirus sequences, e.g. Zika sequences, downstream of the target Flavivirus sequences, e.g. Zika sequences, of the gRNA.

In another embodiment, a target nucleic acid sequence comprises one or more nucleic acid sequences in coding and non-coding nucleic acid sequences of the Flavivirus genome. The target nucleic acid sequence can be located within a sequence encoding structural proteins, non-structural proteins or combinations thereof. The sequences encoding structural proteins comprise nucleic acid sequences encoding a capsid protein (C), precursor viral membrane protein (prM), viral membrane protein (M), envelop protein (E) or combinations thereof. The sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

In one embodiment, a guide oligonucleotide comprises at least five consecutive bases complementary to a nucleic acid sequence, wherein the oligonucleotide specifically hybridizes to a nucleic acid sequence comprising one or more mutations or variants of a Flavivirus, e.g. Zika virus, in vivo or in vitro. In another preferred embodiment, the guide oligonucleotide sequences of the present invention also include variants in which a different base is present at one or more of the nucleotide positions in the compound. For example, if the first nucleotide is an adenosine, variants may be produced which contain thymidine, guanosine or cytidine at this position. This may be done at any of the positions of the oligonucleotide. These compounds are then tested using the methods described herein to determine their ability to edit and correct a BAG function, activity or expression.

In some embodiments, homology, sequence identity or complementarity, between the oligonucleotide and target nucleic acid sequences of a Flavivirus is from about 50% to about 60%. In some embodiments, homology, sequence identity or complementarity, is from about 60%> to about 70%>. In some embodiments, homology, sequence identity or complementarity, is from about 70%> to about 80%>. In some embodiments, homology, sequence identity or complementarity, is from about 80%> to about 90%). In some embodiments, homology, sequence identity or complementarity, is about 90%>, about 92%, about 94%, about 95%, about 96%, about 97%, about 98%, about 99% or about 100%.

In certain embodiments, a gRNA sequence has at least a 50%, 60%, 65% or at least 75% sequence identity to a nucleic acid sequence that is complementary to, or having at least a 50%, 60%, 65% or at least 75% sequence identity to target nucleic acid sequence encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

In certain embodiments, a gRNA sequence has at least a 75% sequence identity to a nucleic acid sequence that is complementary to a target nucleic acid sequence encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

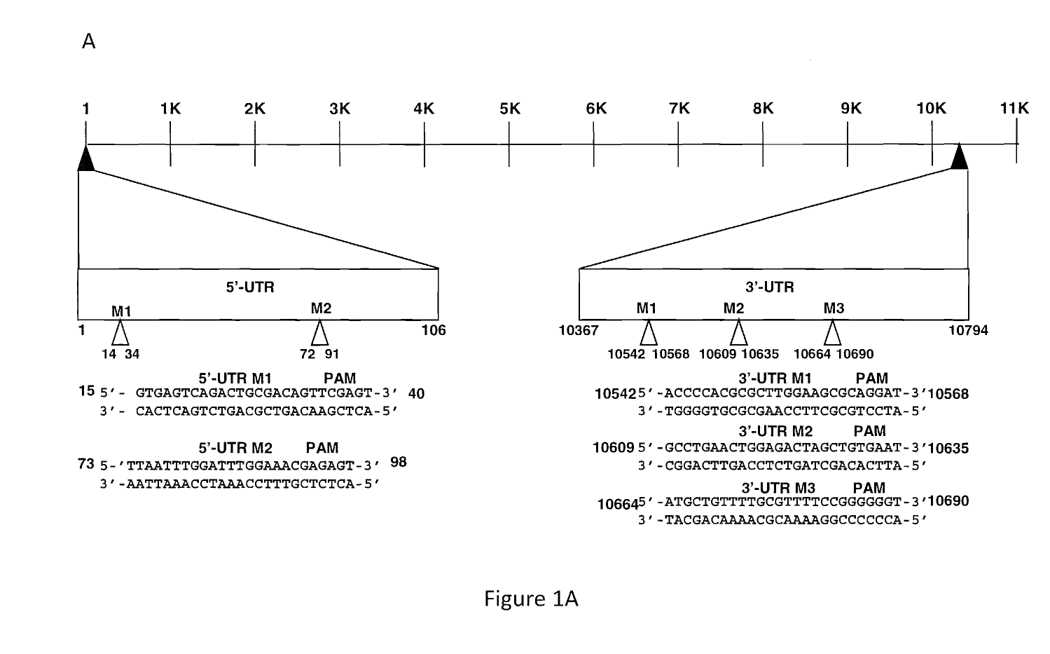

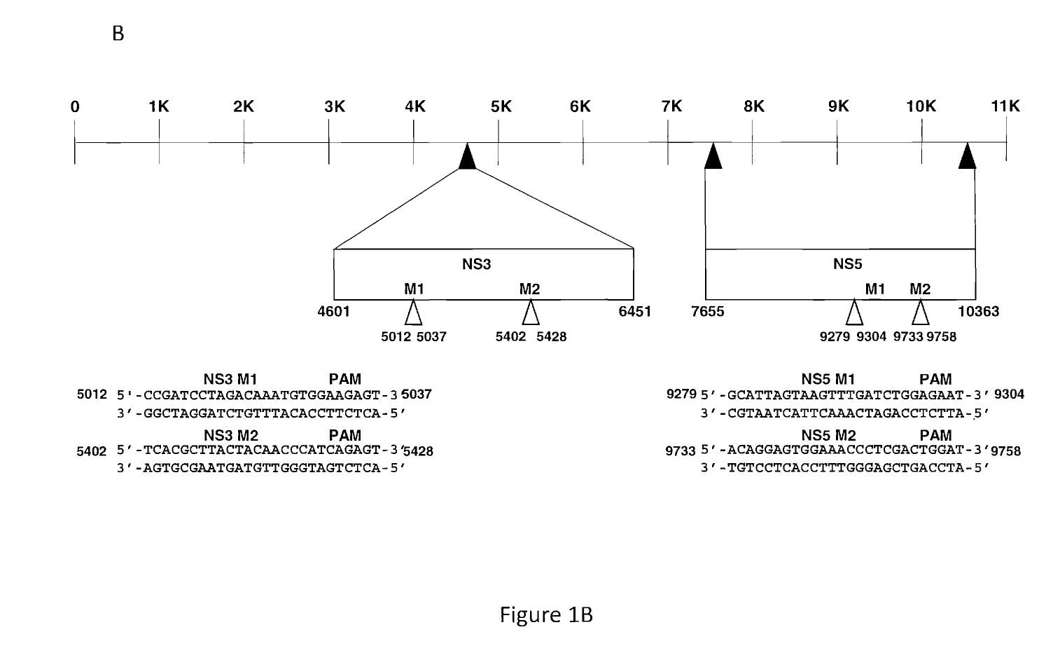

Non-limiting examples of gRNA nucleic acid sequences are shown in FIGS. 1A and 1B and are as follows:

(SEQ ID NO: 1)
5′-GTGAGTCAGACTGCGACAGTTCGAGT-3′
(SEQ ID NO: 2)
3′-CACTCAGTCTGACGCTGACAAGCTCA-5′
(SEQ ID NO: 3)
5′-TTAATTTGGATTTGGAAACGAGAGT-3′
(SEQ ID NO: 4)
3′-AATTAAACCTAAACCTTTGCTCTCA-5
(SEQ ID NO: 5)
5′-ACCCCACGCGCTTGGAAGCGCAGGAT-3′
(SEQ ID NO: 6)
3′-TGGGGTGCGCGAACCTTCGCGTCCTA-5′
(SEQ ID NO: 7)
5′-GCCTGAACTGGAGACTAGCTGTGAAT-3′
(SEQ ID NO: 8)
3′-CGGACTTGACCTCTGATCGACACTTA-5′
(SEQ ID NO: 9)
5′-ATGCTGTTTTGCGTTTTCCGGGGGGT-3′
(SEQ ID NO: 10)
3′-TACGACAAAACGCAAAAGGCCCCCCA-5′
(SEQ ID NO: 11)
5′-CCGATCCTAGACAAATGTGGAAGAGT-3′
(SEQ ID NO: 12)
3′-GGCTAGGATCTGTTTACACCTTCTCA-5′
(SEQ ID NO: 13)
5′-TCACGCTTACTACAACCCATCAGAGT-3′
(SEQ ID NO: 14)
3′-AGTGCGAATGATGTTGGGTAGTCTCA-5′
(SEQ ID NO: 15)
5′-GCATTAGTAAGTTTGATCTGGAGAAT-3′
(SEQ ID NO: 16)
3′-CGTAATCATTCAAACTAGACCTCTTA-5′
(SEQ ID NO: 17)
5′-ACAGGAGTGGAAACCCTCGACTGGAT-3′
(SEQ ID NO: 18)
3′-TGTCCTCACCTTTGGGAGCTGACCTA-5′

Table 1 provides non-limiting examples of RNA-guided Cas9 which cleaves ssRNA targets in the presence of a short PAM-presenting DNA oligonucleotide (PAMmer).

TABLE 1

| Targeted | | corresponding |
|---|---|---|
| region | motif | PAMmer sequence |
| 5′-UTR | 1 | 5′-TCGAGTCTGAAGCGAGAGCT-3′ |
| | | (SEQ ID NO: 19) |
| 5′-UTR | 2 | 5′-GAGAGTTTCTGGTCATGAAA-3′ |
| | | (SEQ ID NO: 20) |
| 3′-UTR | 1 | 5′-CAGGATGGGAAAAGAAGGTG-3 |
| | | (SEQ ID NO: 21) |
| 3′-UTR | 2 | 5′-GTGAATCTCCAGCAGAGGGA-3′ |
| | | (SEQ ID NO: 22) |
| 3′-UTR | 3 | 5′-GGGGGGTCTCCTCTAACCAC-3′ |
| | | (SEQ ID NO: 23) |
| NS3 | 1 | 5′-AAGAGTGATAGGACTCTATG-3′ |
| | | (SEQ ID NO: 24) |
| NS3 | 2 | 5′-CAGAGTCCCTAATTACAATC-3′ |
| | | (SEQ ID NO: 25) |
| NS5 | 1 | 5′-GAGAATGAAGCTCTGATTAC-3′ |
| | | (SEQ ID NO: 26) |
| NS5 | 2 | 5′-CTGGATGGAGCAATTGGGAA-3′ |
| | | (SEQ ID NO: 27) |

In other embodiments, the gRNA sequences have at least a 75% sequence identity to sequences comprising: SEQ ID NOS: 1-18, or combinations thereof. In other embodiments, the gRNA sequences comprise: SEQ ID NOS: 1-18, or combinations thereof.

In other embodiments, the isolated nucleic acid sequences further comprise a short proto-spacer adjacent motif (PAM)-presenting DNA oligonucleotide sequence (PAMmer) wherein the PAMmer oligonucleotides comprise a PAM and additional Zika sequences downstream of the target Zika sequences of the gRNA. In embodiments, the Zika sequences comprise sequences within coding and non-coding nucleic acid sequences. In other embodiments, the nucleic acid sequences are located within nucleic acid sequences encoding structural and non-structural proteins. In certain embodiments, the short PAM-presenting DNA oligonucleotide sequences (PAMmer) have at least a 75% sequence identity to at least one nucleic sequence comprising: SEQ ID NOS: 19-27, or combinations thereof. In other embodiments, the PAMmer sequences comprise at least one of SEQ ID NOS: 19-27, or combinations thereof.

In certain embodiments, an isolated nucleic acid sequence comprises a nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome. In other embodiments, the isolated nucleic acid sequence further comprises one or more PAMmer nucleic acid sequences.

When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the gRNA sequences or in a separate vector.

The gRNA sequences according to the present invention can be complementary to either the sense or anti-sense strands of the target sequences. They can include additional 5′ and/or 3′ sequences that may or may not be complementary to a target sequence. They can have less than 100% complementarity to a target sequence, for example 75%, 60%, 50% complementarity. The gRNA sequences can be employed as a combination of one or more different sequences, e.g., a multiplex configuration. Multiplex configurations can include combinations of two, three, four, five, six, seven, eight, nine, ten, or more different guide RNAs.

Modified or Mutated Nucleic Acid Sequences:

In some embodiments, any of the nucleic acid sequences may be modified or derived from a native nucleic acid sequence, for example, by introduction of mutations, deletions, substitutions, modification of nucleobases, backbones and the like. The nucleic acid sequences include the vectors, gene-editing agents, gRNAs, tracrRNA etc. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂ [known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651).

It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethy1-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethy 1-2-thiouridine; 5-methylaminomethy 1-2-selenouridine; 5-carboxymethylaminomethyluridine; 5-carboxymethylaminomethy 1-2′-O-methyl-uridine; 5-carboxymethylaminomethy 1-2-thiouridine; dihydrouridine; dihydroribosylthymine; 2′-methyladenosine; 2-methyladenosine; N⁶Nmethyladenosine; N6,N6-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopenteny 1)-adenosine; N-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N6-hydroxynorvalylcarbamoyl adenosine; 2-methylthio-N6-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N²,N²-dimethyl guanosine; N², 2′-O-dimethyl guanosine; N²,N², 2′-O-trimethyl guanosine; 2′-O-ribosyl guanosine (phosphate); 7-methyl guanosine; N²;7-dimethyl guanosine; N²; N²; 7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The isolated nucleic acid molecules of the present invention can be produced by standard techniques. For example, polymerase chain reaction (PCR) techniques can be used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Delivery Vehicles

Delivery vehicles as used herein, include any types of molecules for delivery of the compositions embodied herein, both for in vitro or in vivo delivery. Examples, include, without limitation: expression vectors, nanoparticles, colloidal compositions, lipids, liposomes, nanosomes, carbohydrates, organic or inorganic compositions and the like.

In some embodiments, a delivery vehicle is a vector, wherein the vector comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome. In some embodiments, the gene editing agent and the at least one guide oligonucleotide is encoded by the same vector. In other embodiments, a first vector encodes the gene editing agent and a second vector encodes at least one guide oligonucleotide, the guide oligonucleotide being complementary to a target nucleic acid sequence in a Flavivirus genome. In yet another embodiment, a vector encodes at least two or more guide oligonucleotides, the guide oligonucleotides being complementary to the same target nucleic acid sequences and/or different target nucleic acid sequences. In yet another embodiment, a vector encodes one or more gene editing agents. In yet another embodiment a vector encodes one or more gene editing agents, two or more gene editing agents, three or more gene editing agents, and/or one or more guide oligonucleotides, two or more guide oligonucleotides, three or more guide oligonucleotides, or any number and combination of gene editing agents and/or guide oligonucleotides.

Nucleic acids as described herein may be contained in vectors. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized in conjunction with the isolated nucleic acid sequences for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al., J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al., Curr Gene Ther, 5:299-310, 2005). An example non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques. 34: 167-171 (2003). A large variety of such vectors are known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

In some embodiments of the invention, lentiviruses, which are a subclass of retroviruses, are used as viral vectors. Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides.

As opposed to lentiviruses, adenoviral DNA does not integrate into the genome and is not replicated during cell division. Adenovirus and the related AAV would be potential approaches as delivery vectors since they do not integrate into the host's genome. AAV can infect both dividing and non-dividing cells and may incorporate its genome into that of the host cell. For example, because of its potential use as a gene therapy vector, researchers have created an altered AAV called self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell. Otherwise, scAAV carries many characteristics of its AAV counterpart. Methods of the invention may incorporate herpesvirus, poxvirus, alphavirus, or vaccinia virus as a means of delivery vectors.

In some embodiments, the vector is an adenovirus-associated viral vector (AAV), for example, AAV9. The term “AAV vector” means a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, preferably the rep and/or cap genes, but retain functional flanking ITR sequences. Despite the high degree of homology, the different serotypes have tropisms for different tissues. The receptor for AAV1 is unknown; however, AAV1 is known to transduce skeletal and cardiac muscle more efficiently than AAV2. Since most of the studies have been done with pseudotyped vectors in which the vector DNA flanked with AAV2 ITR is packaged into capsids of alternate serotypes, it is clear that the biological differences are related to the capsid rather than to the genomes. Recent evidence indicates that DNA expression cassettes packaged in AAV 1 capsids are at least 1 log 10 more efficient at transducing cardiomyocytes than those packaged in AAV2 capsids. In one embodiment, the viral delivery system is an adeno-associated viral delivery system. The adeno-associated virus can be of serotype I (AAV 1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9). Some skilled in the art have circumvented some of the limitations of adenovirus-based vectors by using adenovirus “hybrid” viruses, which incorporate desirable features from adenovirus as well as from other types of viruses as a means of generating unique vectors with highly specialized properties. For example, viral vector chimeras were generated between adenovirus and adeno-associated virus (AAV). These aspects of the invention do not deviate from the scope of the invention described herein.

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Felgner and Holm, Bethesda Res. Lab. Focus, 11(2):21 (1989) and Maurer, R. A., Bethesda Res. Lab. Focus, 11 (2):25 (1989).

Another delivery method is to use single stranded DNA producing vectors which can produce the expressed products intracellularly. See for example, Chen et al., BioTechniques, 34: 167-171 (2003), which is incorporated herein, by reference, in its entirety.

Expression of the oligonucleotides may be controlled by any promoter/enhancer element known in the art, but these regulatory elements must be functional in the host selected for expression. In some embodiments, the promoter is a tissue specific promoter. Of particular interest are muscle specific promoters, and more particularly, cardiac specific promoters. These include the myosin light chain-2 promoter (Franz et al. (1994) Cardioscience, Vol. 5(4):235-43; Kelly et al. (1995) J. Cell Biol, Vol. 129(2):383-396), the alpha actin promoter (Moss et al. (1996) Biol. Chem., Vol. 271(49):31688-31694), the troponin 1 promoter (Bhavsar et al. (1996) Genomics, Vol. 35(1): 11-23); the Na⁺/Ca²⁺ exchanger promoter (Barnes et al. (1997) J. Biol. Chem., Vol. 272(17): 11510-11517), the dystrophin promoter (Kimura et al. (1997) Dev. Growth Differ., Vol. 39(3):257-265), the alpha7 integrin promoter (Ziober and Kramer (1996) J. Bio. Chem., Vol. 271(37):22915-22), the brain natriuretic peptide promoter (LaPointe et al. (1996) Hypertension, Vol. 27(3 Pt 2):715-22) and the alpha B-crystallin/small heat shock protein promoter (Gopal-Srivastava (1995) J. Mol. Cell. Biol, Vol. 15(12):7081-7090), alpha myosin heavy chain promoter (Yamauchi-Takihara et al. (1989) Proc. Natl. Acad. Sci. USA, Vol. 86(10):3504-3508) and the ANF promoter (LaPointe et al. (1988) J. Biol. Chem., Vol. 263(19):9075-9078).

Other promoters which may be used to control gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78: 1441-1445, 1981), the regulatory sequences of the metallothionein gene (Brinster et al., Nature 296:39-42, 1982); prokaryotic expression vectors such as the 3-lactamase promoter (Villa-Kamaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., Cell 38:639-646, 1984; Ornitz et al., Cold Spring Harbor Symp. Quant. Biol. 50:399-409, 1986; MacDonald, Hepatology 7:425-515, 1987); insulin gene control region which is active in pancreatic beta cells (Hanahan, Nature 315: 115-122, 1985), immunoglobulin gene control region which is active in lymphoid cells (Grosschedl et al., Cell 38:647-658, 1984; Adames et al., Nature 318:533-538, 1985; Alexander et al, Mol. Cell. Biol. 7: 1436-1444, 1987), mouse mammary tumor virus control region which is active in testicular, breast, lymphoid and mast cells (Leder et al., Cell 45:485-495, 1986), albumin gene control region which is active in liver (Pinkert et al., Genes and Devel. 1:268-276, 1987), alpha-fetoprotein gene control region which is active in liver (Krumlauf et al., Mol. Cell. Biol. 5: 1639-1648, 1985; Hammer et al., Science 235:53-58, 1987), alpha 1-antitrypsin gene control region which is active in the liver (Kelsey et al., Genes and Devel. 1: 161-171, 1987), beta-globin gene control region which is active in myeloid cells (Mogram et al, Nature 315:338-340, 1985; Kollias et al, Cell 46:89-94, 1986), myelin basic protein gene control region which is active in oligodendrocyte cells in the brain (Readhead et al., Cell 48:703-712, 1987), myosin light chain-2 gene control region which is active in skeletal muscle (Sani, Nature 314:283-286, 1985), and gonadotropic releasing hormone gene control region which is active in the hypothalamus (Mason et al., Science 234: 1372-1378, 1986).

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly infected reservoirs of the virus, for example, dendritic cells, macrophages, endothelial cells, neuronal cells etc. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CRISPR-associated endonuclease and a guide RNA complementary to a target sequence of a Flavivirus, as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein.

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP).

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions may be formulated as a topical gel for blocking sexual transmission of, for example the Zika virus. The topical gel can be applied directly to the skin or mucous membranes of the male or female genital region prior to sexual activity. Alternatively, or in addition the topical gel can be applied to the surface or contained within a male or female condom or diaphragm.

Regardless of whether compositions are administered as nucleic acids or polypeptides, they are formulated in such a way as to promote uptake by the mammalian cell. Useful vector systems and formulations are described above. In some embodiments, the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more Cas/gRNA vectors. In some embodiments, the methods of the invention can be applied ex vivo. That is, a subject's cells can be removed from the body and treated with the compositions in culture to excise, for example, Zika virus sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments, the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The isolated nucleic acids can be easily delivered to a subject by methods known in the art, for example, methods which deliver siRNA. In some aspects, the Cas may be a fragment wherein the active domains of the Cas molecule are included, thereby cutting down on the size of the molecule. Thus, the Cas9/gRNA molecules can be used clinically, similar to the approaches taken by current gene therapy. In particular, a Cas9/multiplex gRNA stable expression stem cell or iPS cells for cell transplantation therapy as well as vaccination can be developed for use in subjects.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

Methods of Treatment and Pharmaceutical Compositions

The methods of the invention can be expressed in terms of the preparation of a medicament. Accordingly, the invention encompasses the use of the agents and compositions described herein in the preparation of a medicament. The compounds described herein are useful in therapeutic compositions and regimens or for the manufacture of a medicament for use in prevention and/or treatment of Flavivirus infections and/or diseases or conditions associated therewith.

In certain embodiments, a method of eradicating a Flavivirus genome in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

In other embodiments, a method of inhibiting replication of a Flavivirus in a cell or a subject, comprising contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

In some embodiments, the compositions and pharmaceutical compositions thereof, comprise any one or more gene editing agents, any type of gene editing agent, any one or more guide oligonucleotides and any combinations thereof.

The composition of the present invention can also include siRNA, miRNAs (micro-RNAs), shRNAs (short hairpin RNAs), or RNA is (RNA interference) that target critical RNAs (viral mRNA) that translate (non-coding or coding) viral proteins involved with the formation of viral proteins and/or virions. The siRNA, miRNAs, shRNAs, or RNAi can be included in the expression vectors described herein along with the gene editing compositions. These RNA interference approaches are there to suppress the lytic and lysogenic cycles of viruses in order to prevent the virus from continuing to infect new cells. This then allows for ‘zoning in’ on the viral genes with the gene editors herein, in order to not fight continual re-infection. In cases like HIV, there exists FDA approved viral replication inhibitors, and the RNA interference approach is not necessarily needed. However, for most viruses such treatments do not exist, so the RNA interference approach to inhibit replication is useful.

In addition, one or more agents which inhibit or decrease virus replication, infection, etc., and/or alleviate any other symptoms or disorders, that may be associated with the virus infection, e.g. fever, chills, headaches, secondary infections, neurological disorders, can be administered in concert with, or as part of the pharmaceutical composition or at separate times. Concurrent administration of two or more therapeutic agents does not require that the agents be administered at the same time or by the same route, as long as there is an overlap in the time period during which the agents are exerting their therapeutic effect. Simultaneous or sequential administration is contemplated, as is administration on different days or weeks. Accordingly, in embodiments, the compositions may also be administered with another standard therapeutic agent for treatment of a Flavivirus infection. These agents comprise, without limitation, an anti-pyretic agent, anti-inflammatory agent, chemotherapeutic agent, anti-viral agents, enzyme inhibitors, such as small molecule antiviral inhibitors, protease inhibitors, telaprevir, boceprevir, or combinations thereof.

In certain embodiments, the anti-viral agent comprises therapeutically effective amounts of: antibodies, aptamers, adjuvants, anti-sense oligonucleotides, chemokines, cytokines, immune stimulating agents, immune modulating molecules, B-cell modulators, T-cell modulators, NK cell modulators, antigen presenting cell modulators, enzymes, siRNA's, interferon, ribavirin, protease inhibitors, anti-sense oligonucleotides, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, vaccines, or combinations thereof.

The immune-modulating molecules comprise, but are not limited to cytokines, lymphokines, T cell co-stimulatory ligands, etc. An immune-modulating molecule positively and/or negatively influences the humoral and/or cellular immune system, particularly its cellular and/or non-cellular components, its functions, and/or its interactions with other physiological systems. The immune-modulating molecule may be selected from the group comprising cytokines, chemokines, macrophage migration inhibitory factor (MIF; as described, inter alia, in Bernhagen (1998), Mol Med 76(3-4); 151-61 or Metz (1997), Adv Immunol 66, 197-223), T-cell receptors or soluble MHC molecules. Such immune-modulating effector molecules are well known in the art and are described, inter alia, in Paul, “Fundamental immunology”, Raven Press, New York (1989). In particular, known cytokines and chemokines are described in Meager, “The Molecular Biology of Cytokines” (1998), John Wiley & Sons, Ltd., Chichester, West Sussex, England; (Bacon (1998). Cytokine Growth Factor Rev 9(2):167-73; Oppenheim (1997). Clin Cancer Res 12, 2682-6; Taub, (1994) Ther. Immunol. 1(4), 229-46 or Michiel, (1992). Semin Cancer Biol 3(1), 3-15).

Immune cell activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin O and pneumolysin facilitate granzyme B-dependent apoptosis (Browne et al., Mol. Cell Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

The compositions of the present invention can be prepared in a variety of ways known to one of ordinary skill in the art. Regardless of their original source or the manner in which they are obtained, the compositions disclosed herein can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having a Zika viral infection or at risk for contracting a Zika virus infection. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal, and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular, or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

The pharmaceutical compositions may contain, as the active ingredient, nucleic acids and vectors described herein in combination with one or more pharmaceutically acceptable carriers. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. An effective amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

Kits

The compositions described herein can be packaged in suitable containers labeled, for example, for use as a therapy to treat a subject having a flavivirus infection, for example, a Zika virus infection or a subject at risk of contracting for example, a Zika virus infection. The containers can include a composition comprising a nucleic acid sequence, e.g. an expression vector encoding a CRISPR-associated endonuclease, for example, a Cas9 endonuclease, and a guide RNA complementary to a target sequence in a flavivirus virus, or a vector encoding that nucleic acid, and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including at least one composition of the invention, e.g., a nucleic acid sequence encoding a CRISPR-associated endonuclease, for example, a Cas9 endonuclease, and a guide RNA complementary to a target sequence in a Zika virus, or a vector encoding that nucleic acid and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

What is claimed:

1. A composition for eradicating a flavivirus in vitro or in vivo, the composition comprising: an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

2. The composition of claim 1, wherein the Flavivirus comprises: dengue virus, tick-borne encephalitis virus, West Nile virus, yellow fever virus, Japanese encephalitis virus, Kyasanur Forest disease virus, Alkhurma hemorrhagic fever virus, Omsk hemorrhagic fever virus, or Zika virus.

3. The composition of claim 2, wherein the Flavivirus is Zika virus.

4. The composition of claim 1, wherein the target nucleic acid sequence comprises one or more nucleic acid sequences in coding and non-coding nucleic acid sequences of the Flavivirus genome.

5. The composition of claim 1, wherein the target nucleic acid sequence comprises one or more sequences within a sequence encoding structural proteins, non-structural proteins or combinations thereof.

6. The composition of claim 5, wherein the sequences encoding structural proteins comprise nucleic acid sequences encoding a capsid protein (C), precursor viral membrane protein (prM), viral membrane protein (M), envelop protein (E) or combinations thereof.

7. The composition of claim 5, wherein the sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

8. The composition of claim 1, wherein the gRNA sequence has at least a 75% sequence identity to a nucleic acid sequence that is complementary to a target nucleic acid sequence encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

9. The composition of claim 1, wherein the gRNA sequences have at least a 75% sequence identity to sequences comprising: SEQ ID NO: 1-18, or combinations thereof.

10. The composition of claim 9, wherein the gRNA sequences comprise: SEQ ID NO: 1-18, or combinations thereof.

11. The composition of claim 1, further comprising a short proto-spacer adjacent motif (PAM)-presenting DNA oligonucleotide sequence (PAMmer) wherein the PAMmer comprises a PAM and additional Flavivirus nucleic acid sequences downstream of target Flavivirus nucleic acid sequences of the gRNA.

12. The composition of claim 11, wherein a PAMmer oligonucleotide sequence comprises a nucleic acid sequence having at least a 75% sequence identity to at least one nucleic acid sequence comprising: SEQ ID NOS: 19-27, or combinations thereof.

13. The composition of claim 12, wherein the PAMmer has at least one nucleic acid sequence comprising SEQ ID NOS: 19-27, or combinations thereof.

14. An isolated nucleic acid sequence encoding a Clustered Regularly Interspaced Short Palindromic Repeat (CRISPR)-associated endonuclease and at least one guide oligonucleotide, the guide oligonucleotide being complementary to a target nucleic acid sequence in a Flavivirus genome.

15. The isolated nucleic acid sequence of claim 14, wherein the Flavivirus comprises: dengue virus, tick-borne encephalitis virus, West Nile virus, yellow fever virus, Japanese encephalitis virus, Kyasanur Forest disease virus, Alkhurma hemorrhagic fever virus, Omsk hemorrhagic fever virus, or Zika virus.

16. The isolated nucleic acid sequence of claim 15, wherein the Flavivirus is Zika virus.

17. The isolated nucleic acid sequence of claim 14, wherein the target nucleic acid sequence comprises one or more nucleic acid sequences in coding and non-coding nucleic acid sequences of the Flavivirus genome.

18. The isolated nucleic acid sequence of claim 14, wherein the target nucleic acid sequence comprises one or more sequences within a sequence encoding structural proteins, non-structural proteins or combinations thereof.

19. The isolated nucleic acid sequence of claim 18, wherein the sequences encoding structural proteins comprise nucleic acid sequences encoding a capsid protein (C), precursor viral membrane protein (prM), viral membrane protein (M), envelop protein (E) or combinations thereof.

20. The isolated nucleic acid sequence of claim 18, wherein the sequences encoding non-structural proteins comprise nucleic acid sequences encoding: non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

21. The isolated nucleic acid sequence of claim 14, wherein the guide oligonucleotide sequence has at least a 75% sequence identity to a nucleic acid sequence that is complementary to a target nucleic acid sequence encoding a capsid protein (C), precursor viral membrane protein (prM), viral membrane protein (M), envelop protein (E), non-structural protein 1 (NS1), non-structural protein 2A (NS2A), non-structural protein 2B (NS2B), non-structural protein 3 (NS3), non-structural protein 4A (NS4A), non-structural protein 4B (NS4B), non-structural protein 5 (NS5), or combinations thereof.

22. The isolated nucleic acid sequence of claim 14, wherein the guide oligonucleotide sequences have at least a 75% sequence identity to at least one sequence comprising: SEQ ID NO: 1-27, or any combinations thereof.

23. The isolated nucleic acid sequence of claim 22, wherein the guide oligonucleotide sequences comprise: SEQ ID NO: 1-27, or combinations thereof.

24. The isolated nucleic acid sequence of claim 23, further comprising a short proto-spacer adjacent motif (PAM)-presenting DNA oligonucleotide sequence (PAMmer) wherein the PAMmer comprises a PAM and additional Flavivirus nucleic acid sequences downstream of target Flavivirus nucleic acid sequences of the gRNA.

25. The isolated nucleic acid sequence of claim 24, wherein a PAMmer oligonucleotide sequence has at least one nucleic acid sequence comprising SEQ ID NOS: 19-27, or combinations thereof.

26. A vector comprising an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

27. A composition comprising a vector encoding an isolated nucleic acid sequence encoding a gene editing agent and/or at least one guide RNA (gRNA), the gRNA being complementary to a target nucleic acid sequence in a Flavivirus genome.

28. The composition of claim 27, wherein one vector encodes the gene editing agent and a separate vector encodes at least one guide oligonucleotide, the guide oligonucleotide being complementary to a target nucleic acid sequence in a Flavivirus genome.

29. The composition of claim 27, wherein a vector encodes a multiplex of guide oligonucleotide sequences.

30. The composition of claim 27, wherein a vector encodes one or more gene editing agents.

31. The composition of claim 27, wherein the gene editing agent comprises Clustered Regularly Interspaced Short Palindromic Repeat (CRISPR)-associated endonuclease, Argonaute family of endonucleases, zinc-finger nucleases (ZFNs), transcription activator-like effector nucleases (TALENs), meganucleases, other endo- or exo-nucleases, or combinations thereof.

32. The composition of claim 31, wherein the gene editing agent comprises Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments or combinations thereof.

33. A delivery vehicle comprising the composition of claim 1, the isolated nucleic acid sequence of claim 14, the expression vector of claim 26, or the composition of claim 27.

34. A method of eradicating a Flavivirus genome in a cell or a subject, comprising contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.

35. A method of inhibiting replication of a Flavivirus in a cell or a subject, comprising contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Flavivirus genome.
